(19) United States
(12) Patent Application Publication
(10) Pub. No.: US 2010/0152277 A1
(43) Pub. Date: Jun. 17, 2010

(54) DIACYLGLYCEROL ACYLTRANSFERASE ASSAY

(76) Inventors: Katharina Antonia Germania Josepha Maurita De Waepenaert, Vosselaar (BE); Daniëlle Celine Georgette Peeters, Herk-De-Stad (BE); Geert Maria Robert Van Hecke, Turnhout (BE); Peter Guido Ida Vermeulen, Oelegem (BE); Monique Jenny Marie Berwaer, Manhay (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/720,633

(22) PCT Filed: Dec. 13, 2005
(86) PCT No.: PCT/EP2005/056739

§ 371 Date: Jun. 1, 2007

(30) Foreign Application Priority Data
Dec. 22, 2004 (EP) 04106850.3

(51) Int. Cl. A61K 31/4015; C12Q 1/48; A61P 9/00

(52) U.S. Cl. 514/425; 435/15

(57) ABSTRACT

The present invention generally provides a method of measuring the biological activity of diacylglycerol acyltransferase (DGAT). Specifically, the present invention provides a method for rapid, mass screening of compounds which are able to modulate the biological activity of DGAT. More specifically, the present invention provides an assay system for measuring DGAT activity based on the use of particular micelles with the FlashPlate™ technology.

### FIELD OF THE INVENTION

The present invention generally provides a method of measuring the biological activity of diacylglycerol acyltransferase (DGAT). Specifically, the present invention provides a method for rapid, mass screening of compounds which are able to modulate the biological activity of DGAT. More specifically, the present invention provides an assay system for measuring DGAT activity based on the use of particular micelles with the FlashPlate technology.

### BACKGROUND TO THE INVENTION

Triglycerides represent the major form of energy stored in eukaryotes. Disorders or imbalances in triglyceride metabolism are implicated in the pathogenesis of and increased risk for obesity, insulin resistance syndrome and type 11 diabetes, nonalcoholic fatty liver disease and coronary heart disease (see, Lewis, et al, Endocrine Reviews (2002) 23:201 and Malloy and Kane, Adv Intern Med (2001) 47:11 1). Additionally, hypertriglyceridemia is often an adverse consequence of cancer therapy (see, Bast, et al. Cancer Medicine, 5th Ed., (2000) B.C. Decker, Hamilton, Ontario, Calif.).

A key enzyme in the synthesis of triglycerides is acyl CoA:diacylglycerol acyltransferase, or DGAT. DGAT is a microsomal enzyme that is widely expressed in mammalian tissues and that catalyzes the joining of 1,2-diacylglycerol (DAG) and fatty acyl CoA to form triglycerides (TG) at the endoplasmic reticulum (reviewed in Chen and Farese, Trends Cardiovasc Med (2000) 10:188 and Farese, et al, Curr Opin Lipidol (2000) 11:229). It was originally thought that DGAT uniquely controlled the catalysis of the final step of acylation of diacylglycerol to triglyceride in the two major pathways for triglyceride synthesis, the glycerol phosphate and monoacylglycerol pathways. Because triglycerides are considered essential for survival, and their synthesis was thought to occur through a single mechanism, inhibition of triglyceride synthesis through inhibiting the activity of DGAT has been largely unexplored.

Genes encoding mouse DGAT1 and the related human homologs ARGP1 and ARGP2 now have been cloned and characterized (Cases, et al, Proc Natl Acad Sci (1998) 95:13018; Oelkers, et al, J. Biol Chem (1998) 273:26765). The gene for mouse DGAT 1 has been used to create DGAT knock-out mice to better elucidate the function of the DGAT gene.

Unexpectedly, mice unable to express a functional DGAT enzyme (Dgat-/- mice) are viable and still able to synthesize triglycerides, indicating that multiple catalytic mechanisms contribute to triglyceride synthesis (Smith, et al, Nature Genetics (2000) 25:87). Other enzymes that catalyze triglyceride synthesis, for example, DGAT2 and diacylglycerol transacylase, also have been identified (Buhman, J. Biol Chem, supra and Cases, et al, J. Biol Chem (2001) 276:38870). Gene knockout studies in mice have revealed that DGAT2 plays a fundamental role in mammalian triglyceride synthesis and is required for survival. DGAT2 deficient mice are lipopenic and die soon after birth, apparently from profound reductions in substrates for energy metabolism and from impaired permeability barrier function in the skin.(Farese et al. JBC (2004) 279:11767).

Significantly, Dgat-/- mice are resistant to diet-induced obesity and remain lean. Even when fed a high fat diet (21% fat) Dgat-/- mice maintain weights comparable to mice fed a regular diet (4% fat) and have lower total body triglyceride levels. The obesity resistance in Dgat-/- mice is not due to deceased caloric intake, but the result of increased energy expenditure and decreased resistance to insulin and leptin (Smith, et al, Nature Genetics, supra; Chen and Farese, Trends Cardiovasc Med. supra; and Chen, et al, J Clin Invest (2002) 109:1049). Additionally, Dgat-/- mice have reduced rates of triglyceride absorption (Buhman, et al, J. Biol Chem (2002) 277:25474). In addition to improved triglyceride metabolism, Dgat-/- mice also have improved glucose metabolism, with lower glucose and insulin levels following a glucose load, in comparison to wild-type mice (Chen and Farese, Trends Cardiovasc Med. supra).

The finding that multiple enzymes contribute to catalyzing the synthesis of triglyceride from diacylglycerol is significant, because it presents the opportunity to modulate one catalytic mechanism of this biochemical reaction to achieve therapeutic results in an individual with minimal adverse side effects. Compounds that inhibit the conversion of diacylglycerol to triglyceride, for instance by specifically inhibiting the activity of the human homolog of DGAT1, will find use in lowering corporeal concentrations and absorption of triglycerides to therapeutically counteract the pathogenic effects caused by abnormal metabolism of triglycerides in obesity, insulin resistance syndrome and overt type II diabetes, congestive heart failure and atherosclerosis, and as a consequence of cancer therapy.

Because of the ever increasing prevalence of obesity, type II diabetes, heart disease and cancer in societies throughout the world, there is a pressing need in developing new therapies to effectively treat and prevent these diseases. Therefore there is an interest in developing compounds that can potently and specifically inhibit the catalytic activity of DGAT. However, a mass screen for the isolation of specific DGAT inhibitors has not been previously established due to technical difficulties associated with establishment of such an assay.

Conventional DGAT assays have low activities on the orders of pmoles TG/min/mg microsomal protein and are contaminated by the products of several other enzymatic reactions. Furthermore, the product of the DGAT catalyzed reaction is usually resolved by TLC analysis (Cases S., et al, PNAS (1998) 95:13018; Cheng D., et al., Biochem J. (2001) 359:707; Erickson S. K., et al., J. Lipid Res. (1980) 21:930) or by using cumbersome organic solvent extraction procedures (Coleman R. A., et al., Meth. Enzymology (1992) 209:98). Given the multiple steps involved in the extraction procedures and the low throughput of the TLC analysis, neither of the currently available DGAT assays is useful in high throughput screening format.

In a first effort to improve the available DGAT assays Ramharack R. R. and Spahr M. A. (EP 1 219 716 & US 2002/0127627) altered the procedure by using a solvent system comprising a combination of acetone and chloroform. Using such a solvent system the common extraction procedure could be simplified to a 1-step extraction procedure. It is however an object of the present invention to further simplifies the assay to come to a procedure that is more suitable for high throughput screening by eliminating the need for time-consuming extraction steps and provides an assay that can be performed in a single well format.

### SUMMARY OF THE INVENTION

As noted above, the present invention concerns a DGAT assay specifically adapted to allow for rapid, mass screening of compounds based on the use of particular micelles with the FlashPlate™ technology.

Therefore, in a first aspect the present invention provides for a method for measuring DGAT activity said method comprising; contacting micelles comprising at least one DGAT substrate with DGAT comprising microsomes and determine triglyceride production in the thus obtained reaction mixture.

In a particular embodiment of the present invention the triglyceride production is determined using a scintillating solid support system such as for example a flashplate.

The present invention also provides a method to identify whether a test compound is capable to modulate DGAT activity, said method comprising; contacting micelles comprising at least one DGAT substrate with DGAT comprising microsomes in the presence and absence of the test compound and determine triglyceride production in the thus obtained reaction mixtures and wherein a change in TG production in the presence of the test compound indicates that said compound is capable to modulate DGAT activity.

In an alternative embodiment the tryglyceride production in the aforementioned screening assay is determined using a scintillating solid support system such as for example a flashplate.

In a particular embodiment of the present invention the aforementioned screening assays are used to determine the capability of a test compound to inhibit DGAT activity, wherein a decrease in TG production in the presence of the test compound indicates that said compound is a DGAT inhibitor.

It is also an object of the present invention to provide the use of DGAT substrate comprising micelles in a method according to the invention.

The present invention also provides methods for treating or preventing conditions and disorders associated with DGAT, comprising administering to a subject in need thereof a therapeutically effective amount of a compound identified in a screening method according to the invention.

### Description of Sequences

SEQ ID NO:1 is the nucleotide sequence for human DGAT1.
SEQ ID NO:2 is the amino acid sequence for human DGAT1.
SEQ ID NO:3 is the nucleotide sequence for human DGAT2.
SEQ ID NO:4 is the amino acid sequence for human DGAT2.

### BRIEF DESCRIPTION OF THE DRAWINGS

FIG. 1 Effects of inhibitors on DGAT activity using the 384 well FlashPlate™ screening assay.

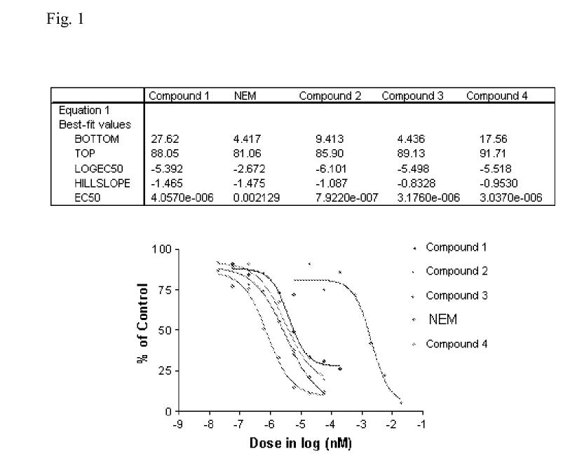

FIG. 2 Effects phophatidylserine (PS) and phosphatidylcholine (PC) in the DGAT substrate comprising nicelles on the DGAT activity in the FlashPlate™ screening assay. At a fixed concentration of PS (3.5 mM) and different concentrations of PC (FIG. 2A) and at a fixed concentration of PC (1.3 mM) and different concentrations of PS (FIG. 2B).

### DETAILED DESCRIPTION OF THE INVENTION

The present invention provides a method for measuring diacylglycerol acetyltransferase (DGAT) biological activity in an assay which allows for rapid and mass screening of the capability of compounds to modulate DGAT activity.

By ‘DGAT’ activity is meant the transfer of coenzyme A activated fatty acids to the 3-position of 1,2-diacylglycerols, forming a triglyceride molecule.

As used herein, the term ‘triglyceride’ (TG, triacylglycerol or neutral fat) refers to a fatty acid triester of glycerol. Triglycerides are typically non-polar and water-insoluble. Phosphoglycerides (or glycerophospholipids) are major lipid components of biological membranes. The fats and oils in animals comprise largely mixtures of triglycerides.

As used herein, the term ‘modulate’ is meant to increase or decrease a function. Preferably, a compound that modulates DGAT activity does so by at least 10%, more preferably by at least 25% and most preferably by at least 50% and can be defined as a ‘modulator’ of DGAT activity.

The method generally includes the steps of combining micelles comprising at least one DGAT substrate with DGAT comprising microsomes, incubate the thus obtained reaction mixture for a predetermined time, stop the reaction and determine the amount of TG produced as an indicator of DGAT activity.

The micelles, comprising the DGAT substrate consists of phospholipids liposomes typically comprising phosphatidylserine or phosphatidylcholine, more particular comprising phosphatidylserine and phosphatidylcholine, preferably with a phosphatidylcholine concentration that is smaller than or equal to the phosphatidylserine concentration, even more particular comprising phosphatidylserine and phosphatidylcholine in a 3:1 molar ratio, most particular comprising phosphatidylserine and phosphatidylcholine in a 3.5:1.3 molar ratio. The DGAT substrates generally used in the methods of the present invention are 1,2-diacylglycerol (DAG), such as for example 1-stearoyl-2-arachidonyl-sn-glycerol or 1,2-dioleoyl-sn-glycerol and a coenzymeA activated fatty acid, such as for example palmitoyl CoA or oleoyl-CoA. In a particular embodiment of the present invention the micelles comprising the DGAT substrate comprise phosphatidylserine and phosphatidylcholine in a 1:1 by weight ratio and 1,2-dioleoyl-sn-glycerol as DGAT substrate. In a preferred embodiment the micelles consist of phosphatidylcholine and phosphatidylserine at 1.3 mM and 3.5 mM respectively with 1.6 mM DAG as substrate. Said DGAT substrate comprising micelles can be prepared as for example provided in Example 3 hereinafter and stored as micelles stock at −20° C. for later use.

The DGAT comprising microsomes as used in the methods of the present invention could either be obtained from insect cell over-expression systems or from tissue microsome preparations, preferably the enzyme source for activity measurements is obtained from insect cell-over expression systems.

Tissue microsome preparations are typically obtained from liver and intestine as for example described by Coleman R. (Coleman R., Diacylglycerol acyltransferase and monoacylglycerol acyltransferase from liver and intestine. Methods in Enzymology 1992; 209:98-104).

In insect cell-overexpression systems, membrane preparations of insect cells (sf9, sf21, or High Five cells) transfected with an appropriate expression vector, such as for example the commercially available Bac-to-Bac Baculovirus expression system, comprising a nucleic acid sequence encoding for a DGAT enzyme, are used. Membrane preparations are obtained using art-known procedures and typically comprise lyses and homogenising the cells using a homogenization device and collecting total cell membranes by ultracentrifugation. The thus obtained membrane preparations can be divided in aliquots and stored with 10% glycerol at −80° C. for later use.

The reaction of DGAT with its substrates is generally initiated by contacting the DGAT comprising microsomes with the micelles as defined hereinbefore, in the presence of a coenzymeA activated fatty acid, in particular in the presence of oleoyl-CoA, wherein optionally, part of said coenzymeA activated fatty acid is detectably labeled. A detectable label as used herein is meant to include radioisotopes such as ¹⁴C or ³H or fluorescent labels such as for example pyrene decanoic acid. It is accordingly an object of the invention to provide the use of radiolabeled or fluorescent labeled coenzymeA activated fatty acids in the methods according to the invention, in particular the use of [¹⁴C]-oleoyl-CoA or (1-pyren-1-yl)decanoyl-CoA. In a more particular embodiment of the present invention the use of [¹⁴C]-oleoyl-CoA.

The reaction mixture is typically incubated at a temperature ranging from room temperature to 37° C. for a predetermined time, such as for example from 5 min.-180 min., more particular at, at least 23° C. for at least 15 min., even more particular at 37° C. for 120 min.

The termination of the reaction of DGAT with its substrates can be accomplished by the addition of an DGAT inhibitor such as for example N-ethylmaleimide, N-(7,10-dimethyl-11-oxo-10,11-dihydro-dibenzo [b,f][1,4]oxazepin-2-yl)-4-hydroxy-benzamide or OT-13540 (Masahiko Ikeda, Chinatsu Suzuki, Yasuhide Inoue: Effects of OT-13540, a potential antiobesity compound, on plasma triglyceride levels in experimental hypertriglyceridemia; XIIIth International Symposium on Atherosclerosis (Kyoto, Japan,) September-October, 2003). Alternatively the reaction is terminated using a denaturing agent such as an alkaline, ethanol comprising stop solution, i.e. 12.5% absolute ethanol, approximately 10% deionized water, approximately 2.5% of 1N NaOH, and approximately 75% of a solution comprising approximately 78.4% isopropanol, approximately 19.6% n-heptane and approximately 2.0% deionized water or chloroform-methanol. In a particular embodiment of the present invention the reaction is terminated using N-ethylmaleimide, N-(7,10-dimethyl-11-oxo-10,11-dihydro-dibenzo[b,f][1,4]oxazepin-2-yl)-4-hydroxy-benzamide or OT-13540, more in particular using N-ethylmaleimide.

### Nucleic Acids

As used in the methods of the present invention, a nucleic acid sequence encoding for a DGAT enzyme is meant to include nucleic acid sequences encoding for either human DGAT1 (SEQ ID No.2) or human DGAT2 (SEQ ID No.4) as well as nucleic acid sequences encoding for other animal, particularly other mammalian, more particularly other primate homologues of human DGAT1 and DGAT2. Said DGAT homologues will typically have at least 50%, for example 60%, 70%, 80%, 90%, 95% or 98% sequence identity to SEQ ID No.2 or SEQ ID No.4. Nucleic acid sequence as used herein includes DNA (including both genomic and cDNA) and RNA. Where nucleic acid according to the invention includes RNA, reference to the sequences shown in the accompanying listings should be construed as reference to the RNA equivalent, with U substituted for T.

Nucleic acid of the invention may be single or double stranded. Single stranded nucleic acids of the invention include anti-sense nucleic acids. Thus it will be understood that reference to SEQ ID NO: 1 or homologues thereof include complementary sequences unless the context is clearly to the contrary.

The cDNA sequence of the DGAT of the invention may be cloned using standard PCR (polymerase chain reaction) cloning techniques. This involves making a pair of primers to 5′ and 3′ ends on opposite strands of SEQ ID NO: 1 or SEQ ID No.3, bringing the primers into contact with mRNA or cDNA obtained from a mammalian cDNA library, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Polynucleotides which are not 100% homologous to the sequence of SEQ ID NO: 1 or SEQ ID No.3 but which encode SEQ ID NO:2 or SEQ ID NO:4 or other polypeptides of the invention can be obtained in a number of ways.

For example, site directed mutagenesis of the sequence of SEQ ID NO: 1 or SEQ ID No.3 may be performed. This is useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides. Further changes may be desirable to represent particular coding changes which are required to provide, for example, conservative substitutions.

Nucleic acids of the invention may comprise additional sequences at the 5′ or 3′ end. For example, synthetic or natural 5′ leader sequences may be attached to the nucleic acid encoding polypeptides of the invention. The additional sequences may also include 5′ or 3′ untranslated regions required for the transcription of nucleic acid of the invention in particular host cells.

In addition, other animal, particularly mammalian (e.g. rats or rabbits), more particularly primate including mouse, homologues of DGAT may be obtained and used in the methods of the present invention. Such sequences may be obtained by making or obtaining cDNA libraries made from dividing cells or tissues or genomic DNA libraries from other animal species, and probing such libraries with probes comprising all or part of SEQ ID NO: 1 or SEQ ID No.3 under conditions of medium to high stringency (for example 0.03M sodium chloride and 0.03M sodium citrate at from about 50° C. to about 60° C.).

### Sequence Identity

The percentage identity of nucleic acid and polypeptide sequences can be calculated using commercially available algorithms which compare a reference sequence with a query sequence. The following programs (provided by the National Center for Biotechnology Information) may be used to determine homologies/identities: BLAST, gapped BLAST, BLASTN and PSI-BLAST, which may be used with default parameters.

The algorithm GAP (Genetics Computer Group, Madison, Wis.) uses the Needleman and Wunsch algorithm to align two complete sequences that maximizes the number of matches and minimizes the number of gaps. Generally, the default parameters are used, with a gap creation penalty=12 and gap extension penalty=4.

Another method for determining the best overall match between a nucleic acid sequence or a portion thereof, and a query sequence is the use of the FASTDB computer program based on the algorithm of Brutlag et al (Comp. App. Biosci., 6; 237-245 (1990)). The program provides a global sequence alignment. The result of said global sequence alignment is in percent identity. Suitable parameters used in a FASTDB search of a DNA sequence to calculate percent identity are: Matrix=Unitary, k-tuple=4, Mismatch penalty=1, Joining Penalty=30, Randomization Group Length=0, Cutoff Score=1, Gap Penalty=5, Gap Size Penalty=0.05, and Window Size=500 or query sequence length in nucleotide bases, whichever is shorter. Suitable parameters to calculate percent identity and similarity of an amino acid alignment are: Matrix=PAM 150, k-tuple=2, Mismatch Penalty=1, Joining Penalty=20, Randomization Group Length=0, Cutoff Score=1, Gap Penalty=5, Gap Size Penalty=0.05, and Window Size=500 or query sequence length in nucleotide bases, whichever is shorter.

### Vectors

Nucleic acid sequences of the present invention may be incorporated into vectors, particularly expression vectors. The vector may be used to replicate the nucleic acid in a compatible host cell. Thus in a further embodiment, the invention provides a method of making polynucleotides of the invention by introducing a polynucleotide of the invention into a replicable vector, introducing the vector into a compatible host cell, and growing the host cell under conditions which bring about replication of the vector. The vector may be recovered from the host cell. Suitable host cells are described below in connection with expression vectors.

Preferably, a polynucleotide of the invention in a vector is operably linked to a control sequence which is capable of providing for the expression of the coding sequence by the host cell, i.e. the vector is an expression vector.

The term “operably linked” refers to a juxtaposition wherein the components described are in a relationship permitting them to function in their intended manner. A control sequence “operably linked” to a coding sequence is ligated in such a way that expression of the coding sequence is achieved under condition compatible with the control sequences.

Suitable vectors can be chosen or constructed, containing appropriate regulatory sequences, including promoter sequences, terminator fragments, polyadenylation sequences, enhancer sequences, marker genes and other sequences as appropriate.

Vectors may be plasmids, viral e.g. phage, phagemid or baculoviral, cosmids, YACs, BACs, or PACs as appropriate. Vectors include gene therapy vectors, for example vectors based on adenovirus, adeno-associated virus, retrovirus (such as HIV or MLV) or alpha virus vectors.

The vectors may be provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used in vitro, for example for the production of RNA or used to transfect or transform a host cell. The vector may also be adapted to be used in vivo, for example in methods of gene therapy. Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others.

Promoters and other expression regulation signals may be selected to be compatible with the host cell for which the expression vector is designed. For example, yeast promoters include S. cerevisiae GAL4 and ADH promoters, S. pombe nmt1 and adh promoter. Mammalian promoters include the metallothionein promoter which can be induced in response to heavy metals such as cadmium. Viral promoters such as the SV40 large T antigen promoter or adenovirus promoters may also be used. All these promoters are readily available in the art.

The vectors may include other sequences such as promoters or enhancers to drive the expression of the inserted nucleic acid, nucleic acid sequences so that the polypeptide is produced as a fusion and/or nucleic acid encoding secretion signals so that the polypeptide produced in the host cell is secreted from the cell.

Vectors for production of polypeptides of the invention of for use in gene therapy include vectors which carry a mini-gene sequence of the invention.

For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Vectors may be transformed into a suitable host cell as described above to provide for expression of a polypeptide of the invention. Thus, in a further aspect the invention provides a process for preparing polypeptides according to the invention which comprises cultivating a host cell transformed or transfected with an expression vector as described above under conditions to provide for expression by the vector of a coding sequence encoding the polypeptides, and recovering the expressed polypeptides. Polypeptides may also be expressed in vitro systems, such as reticulocyte lysate.

A further embodiment of the invention provides host cells transformed or transfected with the vectors for the replication and expression of polynucleotides of the invention. The cells will be chosen to be compatible with the said vector and may for example be bacterial, yeast, insect or mammalian. The host cells may be cultured under conditions for expression of the gene, so that the encoded polypeptide is produced. If the polypeptide is expressed coupled to an appropriate signal leader peptide it may be secreted from the cell into the culture medium. Following production by expression, a polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers

Polynucleotides according to the invention may also be inserted into the vectors described above in an antisense orientation in order to provide for the production of antisense RNA or ribozymes.

### Membrane Preparations

The specifics of preparing such cell membranes as used in the methods of the present invention may in some cases be determined by the nature of the ensuing assay but typically involve harvesting whole cells and disrupting the cell, for example by sonication in ice cold buffer (e.g. 20 mM Tris HCl, 1 mM EDTA, pH 7.4 at 4° C.). The resulting crude cell lysate is subsequently cleared of cell debris by low speed centrifugation, for example at 200×g for 5 min at 4° C. Further clearance and membrane enrichment is finally done using a high speed centrifugation step, such as for example 40,000×g for 20 min at 4° C., and the resulting membrane pellet is washed by suspending in ice cold buffer and repeating the high speed centrifugation step. The final washed membrane pellet is resuspended in assay buffer. Protein concentrations are determined by the method of Bradford (1976) using bovine serum albumin as a standard. The membranes may be used immediately or frozen for later use.

In the methods of the present invention the membranes are incubated with DGAT substrates as described herein before, either in the presence or absence of compounds to be tested for their capability to modulate DGAT activity. The DGAT activity is determined by measuring the TG production, wherein said TG production is typically determined by measuring the incorporation of radiolabeled TG in the micelles of the invention using a scintillating solid support medium such as for example the commercially available FlashPlate™ technology. Data is fit to non-linear curves using GraphPad prism.

In this manner, agonist or antagonist compounds that modulate DGAT activity may be identified. It is a particular object of the present invention to use the membrane preparations in methods to identify compounds that are capable to inhibit DGAT activity, i.e. to identify DGAT antagonists.

### Therapeutic Formulations

Thus the invention further provides novel modulatory agents, in particular antagonists obtained by an assay according to the present invention, and compositions comprising such agents. Agents which bind to the receptor and which may have agonist or antagonist activity may be used in methods of treating diseases whose pathology is characterised by action of the DGAT enzyme, in particular obesity and high triacylglycerol related diseases and such use forms a further aspect of the invention. Disorders or imbalances in triglyceride metabolism are implicated in the pathogenesis of and increased risk for obesity, insulin resistance syndrome and type H diabetes, nonalcoholic fatty liver disease and coronary heart disease (see, Lewis, et al, Endocrine Reviews (2002) 23: 201 and Malloy and Kane, Advlntern Med (2001) 47: 111). Additionally, hypertriglyceridemia is often an adverse consequence of cancer therapy (see, Bast, et al. Cancer Medicine, 5th Ed., (2000) B. C. Decker, Hamilton, Ontario, Calif.).

The present invention also provides methods for treating or preventing a condition or disorder selected from the group consisting of obesity, diabetes, anorexia nervosa, bulimia, cachexia, syndrome X, metabolic syndrome, insulin resistance, hyperglycemia, hyperuricemia, hyperinsulinemia, hypercholesterolemia, hyperlipidemia, dyslipidemia, mixed dyslipidemia, hypertriglyceridemia, nonalcoholic fatty liver disease, atherosclerosis, arteriosclerosis, acute heart failure, congestive heart failure, coronary artery disease, cardiomyopathy, myocardial infarction, angina pectoris, hypertension, hypotension, stroke, ischermia, ischemic reperfusion injury, aneurysm, restenosis, vascular stenosis, solid tumors, skin cancer, melanoma, lymphoma, breast cancer, lung cancer, colorectal cancer, stomach cancer, esophageal cancer, pancreatic cancer, prostate cancer, kidney cancer, liver cancer, bladder cancer, cervical cancer, uterine cancer, testicular cancer and ovarian cancer, comprising administering to a subject in need thereof a therapeutically effective amount of a compound of the invention. For this method and the methods provided below, the compound of the invention will, in some embodiments, be administered in combination with a second therapeutic agent.

The agents may be administered an effective amount of an agent of the invention. Since many of the above-mentioned conditions are chronic and often incurable, it will be understood that “treatment” is intended to include achieving a reduction in the symptoms for a period of time such as a few hours, days or weeks, and to include slowing the progression of the course of the disease.

Such agents may be formulated into compositions comprising an agent together with a pharmaceutically acceptable carrier or diluent. The agent may in the form of a physiologically functional derivative, such as an ester or a salt, such as an acid addition salt or basic metal salt, or an N or S oxide. Compositions may be formulated for any suitable route and means of administration. Pharmaceutically acceptable carriers or diluents include those used in formulations suitable for oral, rectal, nasal, inhalable, topical (including buccal and sublingual), vaginal or parenteral (including subcutaneous, intramuscular, intravenous, intradermal, intrathecal and epidural) administration. The choice of carrier or diluent will of course depend on the proposed route of administration, which, may depend on the agent and its therapeutic purpose. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound as defined above may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline aqueous dextrose, glycerol, ethanol, and the like, to thereby form a solution or suspension. If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc. Actual methods of preparing such dosage forms are known, or will be apparent, to those skilled in this art; for example, see Remington's Pharmaceutical Sciences, Mack Publishing Company, Easton, Pa., 15th Edition, 1975.

The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium, carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the pharmaceutical compositions to be administered may also contain minor amounts of non-toxic auxiliary substances such as wetting or emulsifying agents, pH buffering agents and the like, such as for example, sodium acetate, sorbitan monolaurate, triethanolamine oleate, triethanolamine sodium acetate, etc.

The percentage of active compound contained in such parental compositions is highly dependent on the specific nature thereof, as well as the activity of the compound and the needs of the subject. However, percentages of active ingredient of 0. 1% to 10% in solution are employable, and will be higher if the composition is a solid which will be subsequently diluted to the above percentages. Preferably, the composition will comprise 0.2-2% of the active agent in solution.

This invention will be better understood by reference to the Experimental Details that follow, but those skilled in the art will readily appreciate that these are only illustrative of the invention as described more fully in the claims that follow thereafter. Additionally, throughout this application, various publications are cited. The disclosure of these publications is hereby incorporated by reference into this application to describe more fully the state of the art to which this invention pertains.

### EXAMPLES

The following examples illustrate the invention. Other embodiments will occur to the person skilled in the art in light of these examples.

### Example 1

### Expression of DGAT

DGAT, acyl-CoA:diacylglycerol acyltransferase, is a key enzyme in triglyceride biosynthesis. DGAT catalyses the reaction of acyl residue transfer from fatty acyl-CoA to diacylglycerol to form TAG by using diacylglycerol (DAG) and fatty acyl CoA as its substrates.

human DGAT1 (SEQ ID No.1) was cloned into the pFastBac vector, containing translation start, a FLAG-tag at the N-terminus as described in literature and a viral Kozak sequence (AAX) preceding the ATG to improve expression in insect cells. Since DGAT is a membrane protein, expression was done as described in literature (Cases, S., Smith, S. J., Zheng, Y., Myers H. M., Lear, S. R., Sande, E., Novak, S., Collins, C., Welch, C. B., Lusis, A. J., Erickson, S. K. and Farese, R. V. (1998) Proc. Natl. Acad. Sci. USA 95, 13018-13023.) using SF9 cells.

### EXAMPLE 2

### Preparation of DGAT Membranes

72 h transfected SF9 cells were collected by centrifugation (13000 rpm-15 min-4° C.) and lysed in 2×500 ml lysisbuffer (0.1M Sucrose, 50 mM KCl, 40 mM KH₂PO₄, 30 mM EDTA pH 7.2. Cells were homogenized by cell disruptor. After centrifugation 1380 rpm-15 min-4° C. (SN discarded), pellet was resuspended in 500 ml lysisbuffer and total cell membranes collected by ultracentrifugation at 34000 rpm(100,000 g) for 60 min (4° C.). The collected membranes were resuspended in lysis buffer, divided in aliquots and stored with 10% glycerol at −80° C. until use.

### EXAMPLE 3

### Preparation of the Micelles

### Materials

a) 1,2-dioleoyl-sn-glycerol, 10 mg/ml (DAG)
   evaporate the acetonitrile solution under nitrogen and reconstitute in chloroform at a final concentration of 10 mg/ml.
b) L-α-phosphatidylcholine, 1 mg/ml (PC)
   Dissolve in chloroform at a final concentration of 1 mg/ml and store at 4° C.
c) L-α-phosphatidyl-L-serine, 1 mg/ml (PS)
   Dissolve in chloroform at a final concentration of 1 mg/ml and store at 4° C.

### Method

Add 1 ml DGA to 10 ml of PC and 10 ml of PS in a thick glass recipient. Evaporate under nitrogen and put on ice for 15 minutes. Reconstitute the thus obtained suspension in 10 ml Tris/HCl (10 mM, pH 7.4) by sonication on ice. The sonification process consists of sonification cycles of 10 seconds in the sonification bath followed by 10 seconds cool down on ice and repeating this sonification cycle till a homogeneous solution is obtained (takes about 15 minutes). The thus obtained micelles are stored at −20° C. till later use and contain DAG at a final concentration of 1.61 mM.

To confirm the optimal 1:1 by weight ratio of phosphatidylserine and phosphatidylcholine in the DGAT substrate comprising micelles, we analyzed the effect of different ratios in the DGAT FlashPlate™ assay.

For the different combinations of phosphatidylcholine and phosphatidylserine, separate mixes were made. Aliquots of stocksolutions of dioleoyl-sn-glycerol(10 mg/ml), L-α-phosphatidylcholine (1 mg/ml) and L-α-phosphatidyl-L-serine(1 mg/ml) in chloroform were combined in glass vials and evaporated under nitrogen and put on ice for 15′. Reconstitution was performed in 10 ml Tris/HCl (10 mM, pH 7.4) by sonification on ice. Aliquots were stored at −20° C.

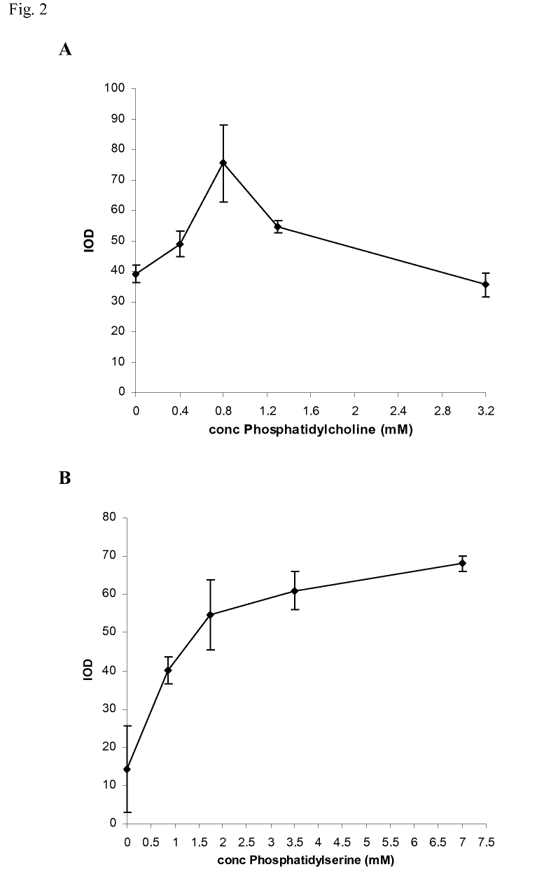

In a first set of experiments the concentration of PC was altered to change the PC:PS ratio. Optimal micelle concentration of phosphatidylcholine for DGAT activity was 0.8 mM (FIG. 2A) with 3.5 mM phoshatidylserine in the micelles. Unfortunately, this concentration resulted in not stable, nor reproducible micelles, indicating that the critical micelle concentration was not reached. Lipids are defined generally on the basis of their solubility properties. They are readily soluble in non-polar solvents and practically insoluble in water. A measure of solubility of amphipathic molecules in water is their critical micelle concentration (CMC). This is defined as the concentration of molecules in free solution in equilibrium with molecules in aggregated form. A typical washing-up liquid contains detergents with a CMC in the mM concentration range (3). On the other hand using higher concentrations then 0.8 mM decreased DGAT activity. We concluded that in this conditions 1.6 mM phosphatidylcholine is optimal for reproducible formation of micelles with acceptable DGAT activity.

This was confirmed in a second set of experiments wherein the concentration of PS was altered to change the PS:PC ratio. Testing out different concentrations of micelle phosphatidylserine in micelles containing 1.6 mM diacylglycerol revealed a nice dose response of DGAT activity up till 3.5 mM, after which almost maximal DGAT activity was reached (FIG. 2B). Using less phosphatidylserine not only decreased activity, but also resulted, similar as for phosphatidylcholine, in less stable and not reproducible micelles. By omitting phosphatidylserine almost all DGAT activity disappeared, indicating that phosphatidylserine is crucial for the activity. We concluded that in this conditions 3.5mM phosphatidylserine is optimal for reproducible formation of micelles with acceptable DGAT activity.

Taking in account not only maximal activity, but also stability and reproducibility in formation of micelles, optimal concentrations are reached for phosphatidylcholine and phosphatidylserine at 1.3 mM and 3.5 mM respectively. In this set up phosphatidylserine appears to be crucial for DGAT activity and phosphatidylcholine for stabilization and reproducibility of micelles.

### Example 4

### DGAT FlashPlate™ Assay

### Materials

a) Assaybuffer
   50 mM Tris-HCl (pH 7.4), 150 mM MgCl₂, 1 mM EDTA, 0.2% BSA.
b) N-ethylmaleimide, 5M
   Dissolve 5 g in to a final volume of 8 ml DMSO 100% and store at −20° C. in aliquots till later use.
c) Substrate mix (for 1,384 well plate=3840μ)
   612 μl micel stock (51 μM final)
   16.6 μl oleoylCoA 9.7 mM
   23 μ1 [³H]-oleoylCoA (49 Ci/mmol, 500 μCi/ml)
   3188.4 μl Tris pH 7.4, 10 mM
d) Enzyme mix (for 1,384 well plate=3520 μl) (5 μg/ml)
   Add 11.73 μl of DGAT membrane stock (1500 μg/ml stock) to 3508 μl assay buffer.
e) Stop mix (for 1,384 well plate=7.68 ml) (250 mM)

Add 384 μl of N-ethylmaleimide (5M) to 3.456 ml DMSO 100%, and further dilute 3.84 ml of said solution with 3.84 ml DMSO 10%.

### Method

DGAT activity in membrane preparations was assayed in 50 mM Tris-HCl (pH 7.4), 150 mM MgCl₂, 1 mM EDTA and 0.2% BSA, containing 50 μM DAG, 32 μg/ml PC/PS and 8.4 μM [³H]-oleoylCoA (at a specific activity of 30 nCi/well) in a final volume of 50 μl in 384-well format using the red shifted Basic Image FlashPlate™ (Perkin Elmer Cat.No. SMP400).

In detail, 10 μl enzyme mix and 10 μl substrate mix were added to 30 μl of assay buffer, optionally in the presence of 1 μl DMSO (blank and controls) or 1 μl of the compound to be tested. This reaction mixture was incubated for 120 minutes at 37° C. and the enzymatic reaction stopped by adding 20 μl of the stop mix. The plates were sealed and the vesicles allowed to settle overnight at room temperature. Plates were centrifuged for 5 minutes at 1500 rpm and measured in Leadseeker.

### DISCUSSION

For the moment no real high throughput compatible assay is commercially available, probably due to the fact that traditional enzymatic assays use vesicle preparations to mimic the natural environment of the enzyme where it is embedded in the membrane.

Traditionally TLC separation or solvent extraction is necessary to separate the radiolabeled DAG or acyl COA from the formed radiolabeled TG. This additional handling step prior to measurement of the formed radiolabeled TG, makes these traditional approaches less suitable for high throughput screening were each step, not only increases the cycle time of the assay but may also affect the reproducibility and consistent readout of the assay.

The DGAT activity screening of the present invention still mimics the natural environment of the enzyme since both DGAT comprising membrane preparations and DGAT substrate comprising micelles are used, but is particularly adapted for mass screening of DGAT activity since it is a single well procedure, eliminating the need to separate the formed radiolabeled TG from the radiolabeled acyl COA. This single well screening format is achieved since the observed radioluminescence only results from the formed radio-active triacylglycerol that comes in close proximity of the flashplate surface, in contrast to the radiolabeled acyl CoA that remains in solution.

To conclude, the present invention provides a platform which is more suitable for high throughput screening by eliminating the need for time-consuming TLC and extraction steps and provides more reproducible and dependable results.

1. A method for measuring DGAT activity said method comprising; contacting micelles comprising at least one DGAT substrate with DGAT comprising microsomes and determine triglyceride production in the thus obtained reaction mixture.

2. A method according to claim 1 wherein micelles comprising the DGAT substrate are selected from;
micelles comprise phosphatidylserine or phosphatidylcholine;
micelles comprising phosphatidylserine and phosphatidylcholine; or
micelles comprising phosphatidylserine and phosphatidylcholine in a 1:1 by weight ratio.

3. A method according to claim 1 wherein the reaction mixture further comprises a coenzymeA activated fatty acid.

4. A method according to claim 3 wherein the coenzymeA activated fatty acid is selected from palmitoyl-CoA or oleoyl-CoA.

5. A method according to claim 3, wherein part of said coenzymeA activated fatty acid is detectably labeled.

6. A method according to 5 wherein part of said coenzymeA activated fatty acid is radiolabeled.

7. A method according to claim 3 wherein the coenzymeA activated fatty acid is oleoyl-CoA and part of said oleoyl-CoA is [³H]-oleoyl-CoA.

8. A method according to claim 1 wherein the DGAT substrate consists of stearoyl-2-arachidonyl-sn-glycerol or 1,2-dioleoyl-sn-glycerol

9. A method according to claim 1 wherein the DGAT comprising microsomes are membrane preparation of insect cells expressing the human DGAT 1 (SEQ ID No.2) protein.

10. A method according to claim 1 wherein the triglyceride production is determined using a scintillating solid support medium.

11. A method to identify whether a test compound is capable to modulate DGAT activity, said method comprising; contacting micelles comprising at least one DGAT substrate with DGAT comprising microsomes in the presence and absence of the test compound and determine triglyceride production in the thus obtained reaction mixtures, and wherein a change in triglyceride production in the presence of the test compound indicates that said compound is capable to modulate DGAT activity.

12. A method according to claim 11 wherein micelles comprising the DGAT substrate are selected from;
micelles comprise phosphatidylserine or phosphatidylcholine;
micelles comprising phosphatidylserine and phosphatidylcholine;
micelles comprising a phosphatidylcholine concentration that is smaller than or equal to the phosphatidylserine concentration;
micelles comprising phosphatidylserine and phosphatidylcholine in a 3:1 molar ratio;
micelles comprising phosphatidylserine and phosphatidylcholine in a 3.5:1.3 molar ratio.

13. A method according to claim 11 wherein the reaction mixture further comprises a coenzymeA activated fatty acid.

14. A method according to claim 13 wherein the coenzymeA activated fatty acid is selected from palmitoyl-CoA or oleoyl-CoA.

15. A method according to claim 13, wherein part of said coenzymeA activated fatty acid is detectably labeled.

16. A method according to claim 15 wherein part of said coenzymeA activated fatty acid is radiolabeled.

17. A method according to any one of claim 13 wherein the coenzymeA activated fatty acid is oleoyl-CoA and part of said oleoyl-CoA is [³H]-oleoyl-CoA.

18. A method according to claim 11 wherein the DGAT substrate consists of stearoyl-2-arachidonyl-sn-glycerol or 1,2-dioleoyl-sn-glycerol

19. A method according to claim 11 wherein the DGAT comprising microsomes are membrane preparation of insect cells expressing the human DGAT1 (SEQ ID No.2) protein.

20. A method according to claim 11 wherein the triglyceride production is determined using a scintillating solid support medium.

21. A method of treating diseases whose pathology is characterised by action of the DGAT enzyme, in particular obesity and high triacylglycerol related diseases, said method comprising administering to a subject in need thereof a therapeutically effective amount of a compound identified using the methods according to claim 11.


SEQUENCE LISTING (4 sequences)

SEQ ID NO: 1 - DNA, 1488 bp, homo sapiens
  CDS (1)..(1488)
atggactacaaggacgacgatgacaagggcgaccgcggcagctcccggcgccggaggacagggtcgcggccctcgagccacggcggcggcgggcctgcggcggcggaagaggaggtgcgggacgccgctgcgggccccgacgtgggagccgcgggggacgcgccagccccggcccccaacaaggacggagacgccggcgtgggcagcggccactgggagctgaggtgccatcgcctgcaggattctttattcagctctgacagtggcttcagcaactaccgtggcatcctgaactggtgtgtggtgatgctgatcttgagcaatgcccggttatttctggagaacctcatcaagtatggcatcctggtggaccccatccaggtggtttctctgttcctgaaggatccttatagctggcccgccccatgcctggttattgcggccaatgtctttgctgtggctgcattccaggttgagaagcgcctggcggtgggtgccctgacggagcaggcgggactgctgctgcacgtggccaacctggccaccattctgtgtttcccagcggctgtggtcttactggttgagtctatcactccagtgggctccctgctggcgctgatggcgcacaccatcctcttcctcaagctcttctcctaccgcgacgtcaactcatggtgccgcagggccagggccaaggctgcctctgcagggaagaaggccagcagtgctgctgccccgcacaccgtgagctacccggacaatctgacctaccgcgatctctactacttcctcttcgcccccaccttgtgctacgagctcaactttccccgctctccccgcatccggaagcgctttctgctgcgacggatccttgagatgctgttcttcacccagctccaggtggggctgatccagcagtggatggtccccaccatccagaactccatgaagcccttcaaggacatggactactcacgcatcatcgagcgcctcctgaagctggcggtccccaatcacctcatctggctcatcttcttctactggctcttccactcctgcctgaatgccgtggctgagctcatgcagtttggagaccgggagttctaccgggactggtggaactccgagtctgtcacctacttctggcagaactggaacatccctgtgcacaagtggtgcatcagacacttctacaagcccatgcttcgacggggcagcagcaagtggatggccaggacaggggtgttcctggcctcggccttcttccacgagtacctggtgagcgtccctctgcgaatgttccgcctctgggcgttcacgggcatgatggctcagatcccactggcctggttcgtgggccgctttttccagggcaactatggcaacgcagctgtgtggctgtcgctcatcatcggacagccaatagccgtcctcatgtacgtccacgactactacgtgctcaactatgaggccccagcggcagaggcc

SEQ ID NO: 2 - protein, 496 aa, homo sapiens
MDYKDDDDKGDRGSSRRRRTGSRPSSHGGGGPAAAEEEVRDAAAGPDVGAAGDAPAPAPNKDGDAGVGSGHWELRCHRLQDSLFSSDSGFSNYRGILNWCVVMLILSNARLFLENLIKYGILVDPIQVVSLFLKDPYSWPAPCLVIAANVFAVAAFQVEKRLAVGALTEQAGLLLHVANLATILCFPAAVVLLVESITPVGSLLALMAHTILFLKLFSYRDVNSWCRRARAKAASAGKKASSAAAPHTVSYPDNLTYRDLYYFLFAPTLCYELNFPRSPRIRKRFLLRRILEMLFFTQLQVGLIQQWMVPTIQNSMKPFKDMDYSRIIERLLKLAVPNHLIWLIFFYWLFHSCLNAVAELMQFGDREFYRDWWNSESVTYFWQNWNIPVHKWCIRHFYKPMLRRGSSKWMARTGVFLASAFFHEYLVSVPLRMFRLWAFTGMMAQIPLAWFVGRFFQGNYGNAAVWLSLIIGQPIAVLMYVHDYYVLNYEAPAAEA

SEQ ID NO: 3 - DNA, 1167 bp, homo sapiens
  CDS (1)..(1167)
atgaagaccctcatagccgcctactccggggtcctgcgcggcgagcgtcaggccgaggctgaccggagccagcgctctcacggaggacctgcgctgtcgcgcgaggggtctgggagatggggcactggatccagcatcctctccgccctccaggacctcttctctgtcacctggctcaataggtccaaggtggaaaagcagctacaggtcatctcagtgctccagtgggtcctgtccttccttgtactgggagtggcctgcagtgccatcctcatgtacatattctgcactgattgctggctcatcgctgtgctctacttcacttggctggtgtttgactggaacacacccaagaaaggtggcaggaggtcacagtgggtccgaaactgggctgtgtggcgctactttcgagactactttcccatccagctggtgaagacacacaacctgctgaccaccaggaactatatctttggataccacccccatggtatcatgggcctgggtgccttctgcaacttcagcacagaggccacagaagtgagcaagaagttcccaggcatacggccttacctggctacactggcaggcaacttccgaatgcctgtgttgagggagtacctgatgtctggaggtatctgccctgtcagccgggacaccatagactatttgctttcaaagaatgggagtggcaatgctatcatcatcgtggtcgggggtgcggctgagtctctgagctccatgcctggcaagaatgcagtcaccctgcggaaccgcaagggctttgtgaaactggccctgcgtcatggagctgacctggttcccatctactcctttggagagaatgaagtgtacaagcaggtgatcttcgaggagggctcctggggccgatgggtccagaagaagttccagaaatacattggtttcgccccatgcatcttccatggtcgaggcctcttctcctccgacacctgggggctggtgccctactccaagcccatcaccactgttgtgggagagcccatcaccatccccaagctggagcacccaacccagcaagacatcgacctgtaccacaccatgtacatggaggccctggtgaagctcttcgacaagcacaagaccaagttcggcctcccggagactgaggtcctggaggtgaactga

SEQ ID NO: 4 - protein, 388 aa, homo sapiens
MKTLIAAYSGVLRGERQAEADRSQRSHGGPALSREGSGRWGTGSSILSALQDLFSVTWLNRSKVEKQLQVISVLQWVLSFLVLGVACSAILMYIFCTDCWLIAVLYFTWLVFDWNTPKKGGRRSQWVRNWAVWRYFRDYFPIQLVKTHNLLTTRNYIFGYHPHGIMGLGAFCNFSTEATEVSKKFPGIRPYLATLAGNFRMPVLREYLMSGGICPVSRDTIDYLLSKNGSGNAIIIVVGGAAESLSSMPGKNAVTLRNRKGFVKLALRHGADLVPIYSFGENEVYKQVIFEEGSWGRWVQKKFQKYIGFAPCIFHGRGLFSSDTWGLVPYSKPITTVVGEPITIPKLEHPTQQDIDLYHTMYMEALVKLFDKHKTKFGLPETEVLEVN